(19) United States
(12) Patent Application Publication
(10) Pub. No.: US 2026/0149045 A1
(43) Pub. Date: May 28, 2026

(54) COMPOUND, ELECTROLYTE FOR RECHARGEABLE LITHIUM BATTERY, AND RECHARGEABLE LITHIUM BATTERY INCLUDING THE SAME

(71) Applicant: SAMSUNG SDI CO., LTD., Yongin-si (KR)

(72) Inventors: Jin Ah SEO, Suwon-si (KR); Eui Soo JEONG, Suwon-si (KR); Sun Dae KIM, Suwon-si (KR); Jin Hyeok LIM, Suwon-si (KR); E Rang CHO, Suwon-si (KR); Bo Mee KWON, Suwon-si (KR); Gi Hun JUNG, Suwon-si (KR); Yeon A KIM, Suwon-si (KR); Jae Kwang KIM, Suwon-si (KR); Sang Hoon KIM, Suwon-si (KR)

(73) Assignee: SAMSUNG SDI CO., LTD., Yongin-si (KR)

(21) Appl. No.: 19/399,868

(22) Filed: Nov. 25, 2025

(30) Foreign Application Priority Data
Nov. 26, 2024 (KR) 10-2024-0171050

(51) Int. Cl. H01M 10/0567; C07D 233/96; H01M 10/052; H01M 10/0569

(52) U.S. Cl. CPC H01M 10/0567; C07D 233/96; H01M 10/052; H01M 10/0569; H01M 2300/004

(57) ABSTRACT

The present disclosure relates to a compound, an electrolyte for a rechargeable lithium battery including the compound, and a rechargeable lithium battery including the electrolyte.

### CROSS-REFERENCE TO RELATED APPLICATION

The present application claims priority to Korean Patent Application No. 10-2024-0171050, filed on Nov. 26, 2024, in the Korean Intellectual Property Office, the entire disclosure of which is incorporated herein by reference.

### BACKGROUND

### 1. Field of the Disclosure

The present disclosure relates to a compound, an electrolyte for a rechargeable lithium battery, and a rechargeable lithium battery including the compound.

### 2. Discussion of Related Art

With increasing presence of electronic devices using batteries such as, e.g., mobile phones, laptop computers, electric vehicles, and the like, the demand for rechargeable batteries with high energy density and high capacity has increased. Accordingly, improving the performance of rechargeable lithium batteries may be advantageous.

A rechargeable lithium battery includes positive and negative electrodes that include active materials capable of intercalation and deintercalation of lithium ions, and an electrolyte, and produces electrical energy through oxidation and reduction reactions when the lithium ions are intercalated/deintercalated into/from the positive and negative electrodes.

As the electrolyte of such rechargeable lithium batteries, an electrolyte in which a lithium salt is dissolved in a non-aqueous organic solvent is used. Rechargeable lithium batteries exhibit battery characteristics through complex reactions between the positive electrode and the electrolyte, between the negative electrode and the electrolyte, and the like. Therefore, the use of a desired electrolyte is a relevant parameter in improving the performance of rechargeable lithium batteries.

### SUMMARY

One example embodiment includes a compound that can provide resistance reduction and gas generation reduction at a high voltage and high temperature in a rechargeable lithium battery, and an electrolyte for a rechargeable lithium battery including the compound.

Another example embodiment includes a rechargeable lithium battery including the electrolyte.

One example embodiment includes a compound of the following Chemical Formula 1.

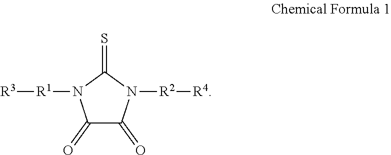

In Chemical Formula 1,
R¹, R², R³, and R⁴ are each as defined in the following description of the disclosure.

Another example embodiment includes an electrolyte for a rechargeable lithium battery, and the electrolyte includes a non-aqueous organic solvent, a lithium salt, and an additive, wherein the additive includes a compound of the following Chemical Formula 1:

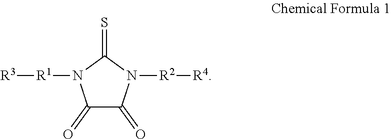

In Chemical Formula 1,
R¹, R², R³, and R⁴ are each as defined in the following description of the disclosure.

Still another example embodiment includes a rechargeable lithium battery including a positive electrode including a positive electrode active material, a negative electrode including a negative electrode active material, and the above-discussed electrolyte.

### BRIEF DESCRIPTION OF THE DRAWINGS

The following drawings attached to the present specification illustrate example embodiments of the present disclosure and further describe aspects and features of the present disclosure together with the detailed description of the present disclosure. Thus, the present disclosure should not be construed as being limited to the drawings, in which:

FIG. 1 is a conceptual diagram schematically showing a rechargeable lithium battery according to one example embodiment of the present disclosure.

FIG. 2 to FIG. 5 are cross-sectional views schematically showing rechargeable lithium batteries according to example embodiments.

### DETAILED DESCRIPTION

In order to fully understand the configurations and effects of the present disclosure, example embodiments of the present disclosure are described with reference to the accompanying drawings. However, it should be understood that the example embodiments disclosed below may be embodied in various forms and modified in various ways without being limited to the example embodiments described herein. The description of the example embodiments is provided only to ensure that the disclosure of the present disclosure is made complete, and to fully inform a person having ordinary skill in the art to which the present disclosure belongs of the scope of the present disclosure.

In the present specification, when any component is referred to as being “on” another component, it means that the component may be formed directly on the other component, or a third component may be interposed therebetween. Also, in the drawings, the thicknesses of components may be exaggerated for the effective description of the technical contents. Throughout the present specification, parts denoted by the same reference numerals denote the same components.

Unless otherwise specified in the present specification, anything indicated in the singular may also include the plural. In addition, unless otherwise particularly stated herein, “A or B” may mean “including A, including B, or including A and B.” As used in the present specification, the term “comprise” and/or “comprising” do not exclude the presence or addition of one or more other components.

In the present specification, the term “combination thereof” may refer to a mixture, laminate, composite, copolymer, alloy, blend, reaction product, and the like of components.

Unless otherwise defined in the present specification, the term “substituted” means that at least one hydrogen in a substituent or compound is replaced with deuterium, a halogen group, a hydroxyl group, an amino group, a C1 to C30 amine group, a nitro group, a C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 fluoroalkyl group, a cyano group, or a combination thereof.

For example, the term “substituted” may mean that at least one hydrogen in a substituent or compound is replaced with deuterium, a halogen group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C10 fluoroalkyl group, or a cyano group. For example, the term “substituted” may mean that at least one hydrogen in a substituent or compound is replaced with deuterium, a halogen group, a C1 to C20 alkyl group, a C6 to C30 aryl group, a C1 to C10 fluoroalkyl group, or a cyano group. Alternatively, the term “substituted” may mean that at least one hydrogen in the substituent or compound is replaced with deuterium, a halogen group, a C1 to C5 alkyl group, a C6 to C18 aryl group, a C1 to C5 fluoroalkyl group, or a cyano group. As an example, the term “substituted” may mean that at least one hydrogen in the substituent or compound is replaced with deuterium, a cyano group, a halogen group, a methyl group, an ethyl group, a propyl group, a butyl group, a phenyl group, a biphenyl group, a terphenyl group, a trifluoromethyl group, or a naphthyl group.

Unless otherwise particularly defined in the present specification, the symbol “*” refers to a moiety that is connected to the same or different atom or chemical formula. Unless specifically mentioned in the chemical formulas described in the present specification, it may be seen that hydrogen is bonded in the structure of the chemical formula.

When the terms “about” or “substantially” are used in this specification in connection with a numerical value, it is intended that the associated numerical value include a tolerance of 10% around the stated numerical value. When ranges are specified, the range includes all values therebetween such as increments of 0.1%.

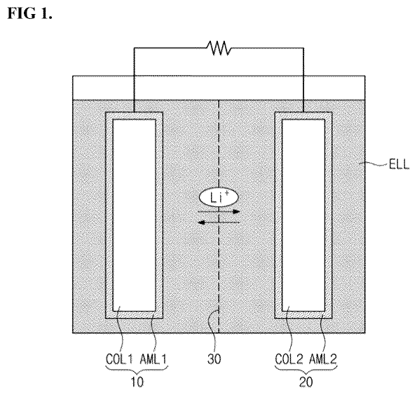

FIG. 1 is a conceptual diagram schematically showing a rechargeable lithium battery according to one example embodiment of the present disclosure. Referring to FIG. 1, the rechargeable lithium battery may include a positive electrode 10, a negative electrode 20, a separator 30, and an electrolyte (ELL).

The positive electrode 10 and the negative electrode 20 may be spaced apart from each other with the separator 30 interposed therebetween. The separator 30 may be disposed between the positive electrode 10 and the negative electrode 20. The positive electrode 10, the negative electrode 20 and the separator 30 may be in contact with the electrolyte (ELL). The positive electrode 10, the negative electrode 20 and the separator 30 may be impregnated with the electrolyte (ELL).

The electrolyte (ELL) may be or include a medium for transferring lithium ions between the positive electrode 10 and the negative electrode 20. In the electrolyte (ELL), the lithium ions may pass through the separator 30 to move toward the positive electrode 10 or the negative electrode 20.

### Positive Electrode 10

A positive electrode 10 for a rechargeable lithium battery may include a current collector (COL1) and a positive electrode active material layer (AML1) formed on the current collector (COL1). The positive electrode active material layer (AML1) includes a positive electrode active material, and may further include a binder and/or a conductive material.

As an example, the positive electrode 10 may further include an additive that may constitute a sacrificial positive electrode.

The content of the positive electrode active material in the positive electrode active material layer (AML1) may range from about 90% by weight to about 99.5% by weight based on 100% by weight of the positive electrode active material layer (AML1). The contents of the binder and conductive material may each range from about 0.5% by weight to about 5% by weight based on 100% by weight of the positive electrode active material layer (AML1).

The binder adheres positive electrode active material particles to each other, and also adheres the positive electrode active material to the current collector (COL1). Representative examples of the binder include at least one of polyvinyl alcohol, carboxymethyl cellulose, hydroxypropyl cellulose, diacetyl cellulose, polyvinyl chloride, carboxylated polyvinyl chloride, polyvinyl fluoride, a polymer containing ethylene oxide, polyvinyl pyrrolidone, polyurethane, polytetrafluoroethylene, polyvinylidene fluoride, polyethylene, polypropylene, a styrene-butadiene rubber, a (meth)acrylated styrene-butadiene rubber, an epoxy resin, a (meth)acrylic resin, a polyester resin, nylon, and the like, but the present disclosure is not limited thereto.

The conductive material may impart conductivity to the electrodes, and any material may be used as long as the conductive material is electronically conductive without causing adverse chemical changes in the battery to be formed. Examples of the conductive material include carbon-based materials such as at least one of natural graphite, artificial graphite, carbon black, acetylene black, Ketjen black, carbon fibers, carbon nanofibers, carbon nanotubes, and the like; metal-based materials in the form of metal powder or metal fibers containing at least one of copper, nickel, aluminum, silver, and the like; conductive polymers such as polyphenylene derivatives and the like; or a mixture thereof.

Al may be used as the current collector (COL1), but the present disclosure is not limited thereto.

### Positive Electrode Active Material

As the positive electrode active material in the positive electrode active material layer (AML1), a compound capable of reversible intercalation and deintercalation of lithium (lithiated intercalation compound) may be used. For example, at least one of a composite oxide of lithium and a metal such as or including at least one of cobalt, manganese, nickel, and a combination thereof may be used.

The composite oxide may be or include a lithium transition metal composite oxide, and examples thereof include at least one of a lithium nickel-based oxide, a lithium cobalt-based oxide, a lithium manganese-based oxide, a lithium iron phosphate-based compound, a cobalt-free nickel-manganese-based oxide, or a combination thereof.

As an example, a compound represented by any one of the following chemical formulas may be used: LiₐA_{1-b}X_{b}O_{2-c}D_{c} (0.90≤a≤1.8, 0≤b≤0.5, 0≤c≤0.05); LiₐMn_{2-b} X_{b}O_{4-c}D_{c} (0.90≤a≤1.8, 0≤b≤0.5, 0≤c≤0.05); LiₐNi_{1-b-c}Co_{b}X_{c}O_{2-α}D_{α} (0.90≤a≤1.8, 0≤b≤0.5, 0≤c≤0.5, 0≤a≤2); LiₐNi_{1-b-c}Mn_{b}X_{c}O_{2-α}D_{α} (0.90≤a≤1.8, 0≤b≤0.5, 0≤c≤0.5, 0≤a≤2); LiₐNi_{b}Co_{c}L¹_{d}GₑO₂ (0.90≤a≤1.8, 0≤b≤0.9, 0≤c 0.5, 0≤d≤0.5, 0≤e≤0.1); LiₐNiG_{b}O₂(0.90≤a≤1.8, 0.001≤b≤0.1); LiₐCoG_{b}O₂(0.90≤a≤1.8, 0.001 K b≤0.1); LiₐMn_{1-b}G_{b}O₂ (0.90≤a≤1.8, 0.001≤b≤0.1); LiₐMn₂GbO₄ (0.90≤a≤1.8, 0.001≤b≤0.1); LiₐMn_{1-g}G_{g}PO₄ (0.90≤a≤1.8, 0≤g≤0.5); Li_{(3-f)}Fe₂(PO₄)₃(0≤f≤2); LiₐFePO₄ (0.90≤a≤1.8).

In the above chemical formulas, A is or includes at least one of Ni, Co, Mn, or a combination thereof, X is or includes at least one of Al, Ni, Co, Mn, Cr, Fe, Mg, Sr, V, a rare earth element, or a combination thereof, D is or includes at least one of O, F, S, P, or a combination thereof, G is or includes at least one of Al, Cr, Mn, Fe, Mg, La, Ce, Sr, V, or a combination thereof, and L¹ is or includes at least one of Mn, Al, or a combination thereof.

As an example, the positive electrode active material may be or include a high-nickel positive electrode active material in which the content of nickel is about 80 mol % or more, about 85 mol % or more, about 90 mol % or more, about 91 mol % or more, or about 94 mol % or more, and about 99 mol % or less, based on 100 mol % of metals excluding lithium in the lithium transition metal composite oxide. The high-nickel positive electrode active material may achieve high capacity, and thus may be applied to high-capacity, high-density rechargeable lithium batteries.

### Negative Electrode 20

A negative electrode 20 for a rechargeable lithium battery includes a current collector (COL2) and a negative electrode active material layer (AML2) disposed on the current collector (COL2). The negative electrode active material layer (AML2) includes a negative electrode active material, and may further include a binder and/or a conductive material.

For example, the negative electrode active material layer (AML2) may include about 90% by weight to about 99% by weight of the negative electrode active material, about 0.5% by weight to about 5% by weight of the binder, and about 0% by weight to about 5% by weight of the conductive material.

The binder adheres negative electrode active material particles to each other, and also adheres the negative electrode active material to the current collector (COL2). Anon-aqueous binder, an aqueous binder, a dry binder, or a combination thereof may be used as the binder.

The non-aqueous binder includes at least one of polyvinyl chloride, carboxylated polyvinyl chloride, polyvinyl fluoride, an ethylene propylene copolymer, polystyrene, polyurethane, polytetrafluoroethylene, polyvinylidene fluoride, polyethylene, polypropylene, polyamideimide, polyimide, or a combination thereof.

The aqueous binder may be or include at least one of styrene-butadiene rubber, (meth)acrylated styrene-butadiene rubber, (meth)acrylonitrile-butadiene rubber, (meth)acrylic rubber, butyl rubber, a fluoroelastomer, polyethylene oxide, polyvinyl pyrrolidone, polyepichlorohydrin, polyphosphazene, poly(meth)acrylonitrile, an ethylene propylene diene copolymer, polyvinyl pyridine, chlorosulfonated polyethylene, latex, a polyester resin, a (meth)acrylic resin, a phenol resin, an epoxy resin, polyvinyl alcohol, and a combination thereof.

When the aqueous binder is used as the negative electrode binder, the aqueous binder may further include a cellulose-based compound capable of imparting viscosity. As the cellulose-based compound, one or more types of carboxymethyl cellulose, hydroxypropylmethyl cellulose, methyl cellulose, or alkali metal salts thereof may be mixed and used. At least one of Na, K or Li may be used as the alkali metal.

The dry binder is a fiberizable polymeric material, and may be or include, for example, at least one of polytetrafluoroethylene, polyvinylidene fluoride, a polyvinylidene fluoride-hexafluoropropylene copolymer, polyethylene oxide, or a combination thereof.

The conductive material may impart conductivity to the electrodes, and any material may be used as long as the conductive material is electronically conductive without causing adverse chemical changes in the battery to be formed. Examples of the conductive material include carbon-based materials such as at least one of natural graphite, artificial graphite, carbon black, acetylene black, Ketjen black, carbon fibers, carbon nanofibers, carbon nanotubes, and the like; metal-based materials in the form of metal powder or metal fibers containing at least one of copper, nickel, aluminum, silver, and the like; conductive polymers such as polyphenylene derivatives and the like; or a mixture thereof.

A current collector including at least one of copper foil, nickel foil, stainless steel foil, titanium foil, a nickel foam, a copper foam, a polymer substrate coated with a conductive metal, and a combination thereof may be used as the current collector (COL2).

### Negative Electrode Active Material

The negative electrode active material in the negative electrode active material layer (AML2) includes at least one of a material capable of reversible intercalation/deintercalation of lithium ions, a lithium metal, an alloy of lithium and a metal, a material capable of doping and dedoping lithium, or a transition metal oxide.

The material capable of reversible intercalation/deintercalation of lithium ions may include a carbon-based negative electrode active material, for example, crystalline carbon, amorphous carbon, or a combination thereof. Examples of the crystalline carbon include graphite such as amorphous, plate-like, flaky, spherical, or fibrous natural or artificial graphite, and examples of the amorphous carbon include at least one of soft carbon or hard carbon, mesophase pitch carbide, calcined coke, and the like.

As the alloy of lithium and a metal, an alloy of lithium and a metal such as or including at least one of Na, K, Rb, Cs, Fr, Be, Mg, Ca, Sr, Si, Sb, Pb, In, Zn, Ba, Ra, Ge, Al, and Sn may be used.

As the material capable of doping and dedoping lithium, a Si-based negative electrode active material or a Sn-based negative electrode active material may be used. The Si-based negative electrode active material may be or include at least one of silicon, a silicon-carbon composite, SiOₓ (0<x<2), a Si-Q alloy (wherein Q is or includes at least one of an alkali metal, an alkaline earth metal, a Group 13 element, a Group 14 element (excluding Si), a Group 15 element, a Group 16 element, a transition metal, a rare earth element, and a combination thereof), or a combination thereof. The Sn-based negative electrode active material may be or include at least one of Sn, SnO₂, a Sn-based alloy, or a combination thereof.

The silicon-carbon composite may be or include a composite of silicon and amorphous carbon. According to one example embodiment, the silicon-carbon composite may be in the form of silicon particles which surfaces are coated with amorphous carbon. For example, the silicon-carbon composite may include a secondary particle (core) in which silicon primary particles are assembled, and an amorphous carbon coating layer (shell) disposed on the surface of the secondary particle. The amorphous carbon may also be located between the silicon primary particles, and for example, the silicon primary particles may be coated with amorphous carbon. The secondary particles may be dispersed in an amorphous carbon matrix.

The silicon-carbon composite may further include crystalline carbon. For example, the silicon-carbon composite may include a core including crystalline carbon and silicon particles, and an amorphous carbon coating layer disposed on the surface of the core.

The Si-based negative electrode active material or the Sn-based negative electrode active material may be used in combination with a carbon-based negative electrode active material.

### Separator 30

A separator 30 may be present between the positive electrode 10 and the negative electrode 20 depending on the type of rechargeable lithium battery. As the separator 30, at least one of polyethylene, polypropylene, polyvinylidene fluoride, or a multi-layer film of two or more layers thereof may be used. Of course, a mixed multi-layer film such as at least one of a polyethylene/polypropylene two-layer separator, a polyethylene/polypropylene/polyethylene three-layer separator, a polypropylene/polyethylene/polypropylene three-layer separator, and the like may also be used.

The separator 30 may include a porous substrate and a coating layer including an organic material, an inorganic material, or a combination thereof and disposed on one surface, or on both surfaces, of the porous substrate.

The porous substrate may be or include a polymer film formed of or including any one polymer such as or including at least one of polyolefins such as polyethylene, polypropylene, and the like, polyesters such as polyethylene terephthalate, polybutylene terephthalate, polyethylene naphthalate and the like, polyacetal, polyamide, polyimide, polycarbonate, polyether ketone, polyarylether ketone, polyetherimide, polyamideimide, polybenzimidazole, polyether sulfone, polyphenylene oxide, a cyclic olefin copolymer, polyphenylene sulfide, glass fibers, Teflon, and polytetrafluoroethylene, or a copolymer or mixture of two or more thereof.

The organic material may include a polyvinylidene fluoride-based polymer or a (meth)acrylic polymer.

The inorganic material may include inorganic particles such as or including at least one of Al₂O₃, SiO₂, TiO₂, SnO₂, CeO₂, MgO, NiO, CaO, GaO, ZnO, ZrO₂, Y₂O₃, SrTiO₃, BaTiO₃, Mg(OH)₂, boehmite, and a combination thereof, but the present disclosure is not limited thereto.

The organic and inorganic materials may be present as a mixture in one coating layer, or may be present in a form in which a coating layer including an organic material and a coating layer including an inorganic material are laminated.

### Electrolyte (ELL)

An electrolyte (ELL) for a rechargeable lithium battery includes a non-aqueous organic solvent and a lithium salt.

The non-aqueous organic solvent constitutes a medium through which ions involved in the electrochemical reaction of the battery may move.

The non-aqueous organic solvent may be or include at least one of a carbonate-based, ester-based, ether-based, ketone-based, or alcohol-based solvent, an aprotic solvent, or a combination thereof.

As the carbonate-based solvent, at least one of dimethyl carbonate (DMC), diethyl carbonate (DEC), dipropyl carbonate (DPC), methyl propyl carbonate (MPC), ethyl propyl carbonate (EPC), ethyl methyl carbonate (EMC), ethylene carbonate (EC), propylene carbonate (PC), butylene carbonate (BC), and the like may be used.

As the ester-based solvent, at least one of methyl acetate, ethyl acetate, n-propyl acetate, dimethyl acetate, methyl propionate, ethyl propionate, decanolide, mevalonolactone, valerolactone, caprolactone, and the like may be used.

As the ether-based solvent, at least one of dibutyl ether, tetraglyme, diglyme, dimethoxyethane, 2-methyltetrahydrofuran, 2,5-dimethyltetrahydrofuran, tetrahydrofuran, and the like may be used. Also, cyclohexanone and the like may be used as the ketone-based solvent. Ethyl alcohol, isopropyl alcohol, and the like may be used as the alcohol-based solvent. At least one of nitriles such as R—CN (where R is a linear, branched, or cyclic hydrocarbon group having 2 to 20 carbon atoms and may include a double bond, an aromatic ring, or an ether group) and the like; amides such as dimethylformamide and the like; dioxolanes such as 1,3-dioxolane, 1,4-dioxolane, and the like; and sulfolanes, may be used as the aprotic solvent.

The non-aqueous organic solvents may be used alone, or in combination of two or more solvents.

Also, when the carbonate-based solvent is used, a cyclic carbonate and a chain carbonate may be used in combination, and the cyclic carbonate and the chain carbonate may be mixed in a volume ratio in a range of about 1:1 to about 1:9.

The lithium salt is a material that dissolves in an organic solvent, and thus constitutes a source of lithium ions in the battery, thereby allowing the basic operation of a rechargeable lithium battery, and promotes the movement of lithium ions between the positive and negative electrodes. Representative examples of the lithium salt may include at least one or more of LiPF₆, LiBF₄, LiSbF₆, LiAsF₆, LiClO₄, LiAlO₂, LiAlCl₄, LiPO₂F₂, LiCl, LiI, LiN(SO₃C₂F₅)₂, Li(FSO₂)₂N (lithium bis(fluorosulfonyl)imide (LiFSI), LiC₄F₉SO₃, LiN(CₓF₂ₓ₊₁SO₂)(C_{y}F_{2y+1}SO₂) (where x and y are integers in a range from 1 to 20), lithium trifluoromethane sulfonate, lithium tetrafluoroethanesulfonate, lithium difluorobis(oxalato)borate (LiDFOB), lithium difluorobis(oxalato)phosphate (LiDFBOP), and lithium bis(oxalato) borate (LiBOB).

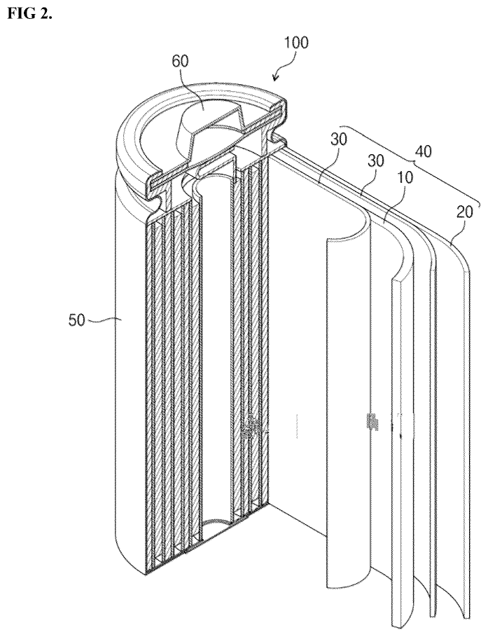

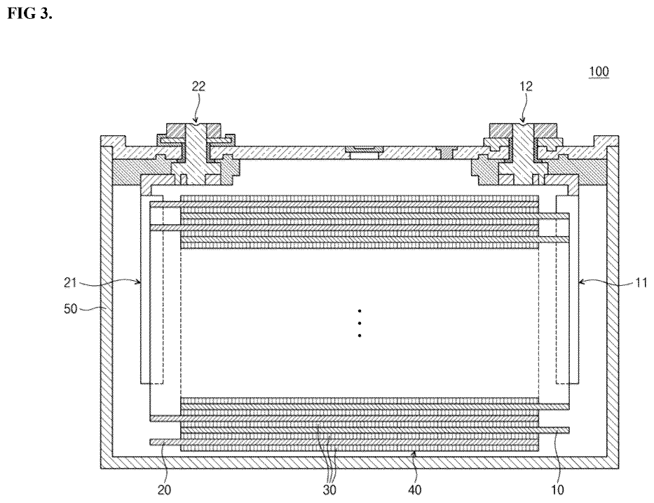

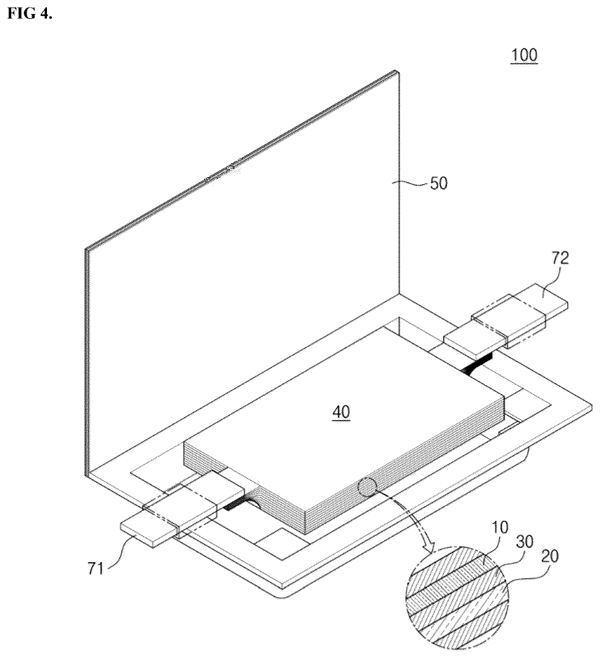

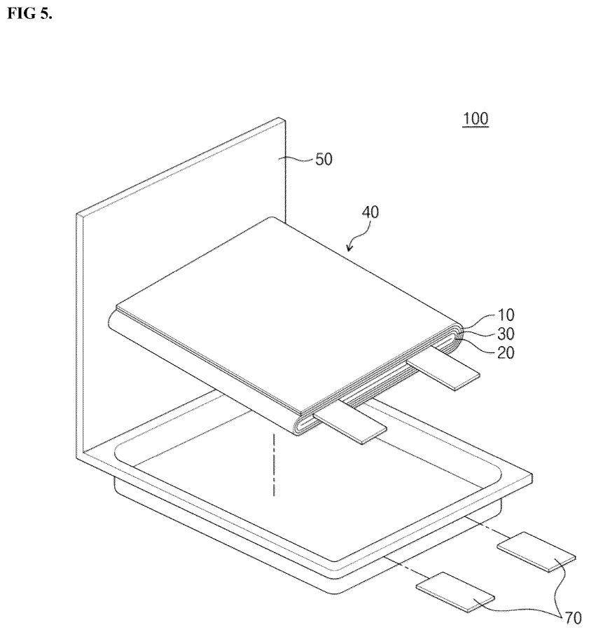

Rechargeable lithium battery Rechargeable lithium batteries may be classified into cylindrical, prismatic, pouch-type, and coin-type rechargeable lithium batteries depending on the type of rechargeable lithium battery. FIG. 2 to FIG. 5 are diagrams schematically showing rechargeable lithium batteries according to example embodiments. The rechargeable lithium batteries can be said to be cylindrical, prismatic, and pouch-type batteries, as shown in FIG. 2, FIG. 3, FIG. 4 and FIG. 5, respectively. Referring to FIG. 2 to FIG. 4, a rechargeable lithium battery 100 may include an electrode assembly 40 having a separator 30 interposed between a positive electrode 10 and a negative electrode 20, and a case 50 in which the electrode assembly 40 is built. The positive electrode 10, the negative electrode 20, and the separator 30 may be impregnated with an electrolyte (not shown). The rechargeable lithium battery 100 may include a sealing member 60 configured to seal the case 50 as shown in FIG. 2. Also, as shown in FIG. 3, the rechargeable lithium battery 100 may include a positive electrode lead tab 11, a positive electrode terminal 12 connected to the positive electrode lead tab 11, a negative electrode lead tab 21, and a negative electrode terminal 22 connected to the negative electrode lead tab 21. As shown in FIG. 4 and FIG. 5, the rechargeable lithium battery 100 may include electrode tab 70 illustrated in FIG. 5, or a positive electrode tab 71 and a negative electrode tab 72 illustrated in FIG. 4, the electrode tabs 70/71/72 forming electric paths configured to conduct current formed in the electrode assembly 40 to the outside of the battery 100.

Hereinafter, a compound according to example embodiments of the present disclosure is described in more detail.

The compound is represented by the following Chemical Formula 1.

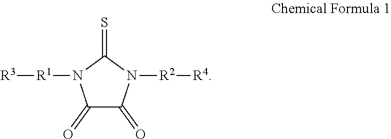

In Chemical Formula 1,
R¹ and R² each independently is or includes a single bond, or a substituted or unsubstituted C1 to C5 alkylene group, and
R³ and R⁴ each independently is or includes a substituted or unsubstituted C5 to C20 cycloalkyl group, a substituted or unsubstituted C6 to C20 aryl group, a substituted or unsubstituted C7 to C20 arylalkyl group, a substituted or unsubstituted C2 to C20 alkenyl group, or a substituted or unsubstituted C2 to C20 alkynyl group.

By using the compound as an additive of an electrolyte of a rechargeable lithium battery, it is possible to reduce a resistance increase rate and a gas generation rate and increase a capacity retention rate after high-temperature storage in a rechargeable lithium battery including a positive electrode active material, for example, a high nickel-based positive electrode active material having a high nickel content, and by providing a low resistance increase rate and a high capacity retention rate at a high temperature, it is possible to increase the lifetime of the battery under high-voltage and high-temperature conditions. In some cases, the compound forms a film on an electrode plate when used as an additive of the electrolyte, and the high temperature stability of the battery can be improved by including a sulfur element in the film, thereby increasing the lifetime of the battery, but the present disclosure is not limited thereto. Here, the high voltage may be about 4.2 V or higher.

In an example, R¹ and R² in Chemical Formula 1 may be or include a single bond or a methylene group.

In an example, R³ and R⁴ in Chemical Formula 1 may each independently be a substituted or unsubstituted C5 to C10 cycloalkyl group, a substituted or unsubstituted C6 to C10 aryl group, a substituted or unsubstituted C7 to C10 arylalkyl group, a substituted or unsubstituted C2 to C10 alkenyl group, or a substituted or unsubstituted C2 to C10 alkynyl group.

For example, the substituted or unsubstituted C5 to C10 cycloalkyl group may be or include a cyclohexyl group or a cyclopentyl group.

For example, the substituted or unsubstituted C6 to C10 aryl group may be or include a phenyl group or a naphthyl group.

For example, the substituted or unsubstituted C2 to C10 alkenyl group may be or include a substituted or unsubstituted C2 to C5 alkenyl group such as a vinyl group or an allyl group.

For example, the substituted or unsubstituted C2 to C10 alkynyl group may be or include a substituted or unsubstituted C2 to C5 alkynyl group, such as propynyl groups including an ethynyl group, a 1-propynyl group, a 2-propynyl group, and the like.

In an example, the compound of Chemical Formula 1 may include one or more of the following Chemical Formula 1-1 to 1-7.

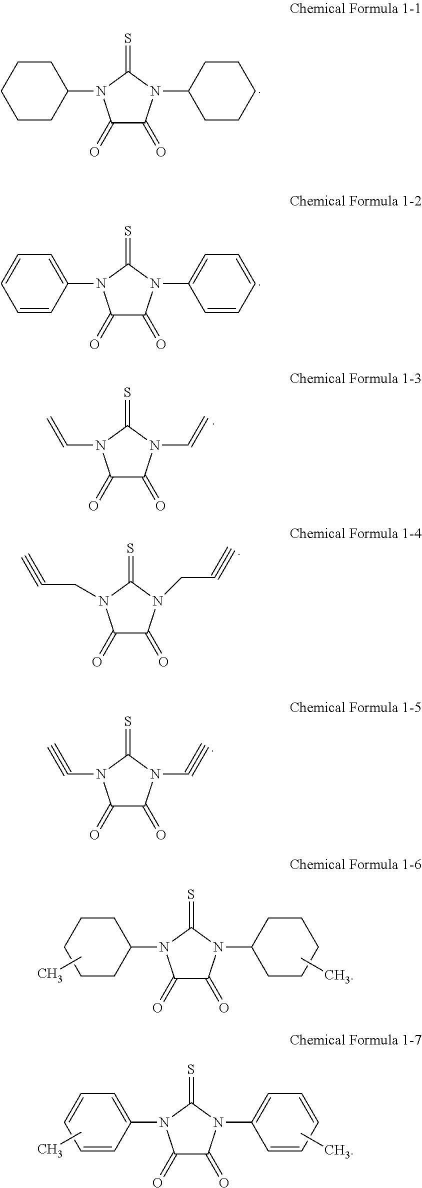

The compound of Chemical Formula 1 may be prepared by conventional methods known to those skilled in the art.

For example, an electrolyte for a rechargeable lithium battery according to example embodiments of the present disclosure is described in more detail below.

The electrolyte for a rechargeable lithium battery according to one example embodiment includes the non-aqueous organic solvent, a lithium salt, and an additive, in which the additive includes the compound of Chemical Formula 1.

The electrolyte may be prepared by dissolving the lithium salt in the non-aqueous organic solvent, adding the additive of Chemical Formula 1, and then performing a mixing process. Processes of mixing electrolytes are widely known in the field of preparing electrolytes, and those skilled in the art may selectively use such processes as desired.

The non-aqueous organic solvent according to one example embodiment of the present disclosure may include one or more of the above non-aqueous organic solvents.

In one example, the non-aqueous organic solvent may be or include a mixture containing ethylene carbonate (EC):ethyl methyl carbonate (EMC):dimethyl carbonate (DMC) in a volume ratio of about 10 to 30:10 to 30:40 to 80. Here, the volume ratio is a value based on a total of 100% by volume of ethylene carbonate (EC):ethyl methyl carbonate (EMC):dimethyl carbonate (DMC). In the above range, it is possible to implement the effect of the additive, and the lifetime of the battery can be further increased under high voltage and high temperature conditions in a rechargeable lithium battery including a positive electrode active material with a high nickel content, which is described below.

The lithium salt according to one example embodiment of the present disclosure may include at least one or more of LiPF₆, LiClO₄, LiBF₄, lithium bis(fluorosulfonyl)imide (LiFSI), lithium bis(trifluoromethanesulfonyl)imide (LiTFSI), LiSO₃CF₃, LiBOB, LiDFOB, LiDFBOP, LiTFOP, LiPO₂F₂, LiSbF₆, LiAsF₆, LiAlO₂, LiAlCl₄, LiCl, LiI, LiN(SO₃C₂F₅)₂, Li(FSO₂)₂N, and LiC₄F₉SO₃. According to one example embodiment, LiPF₆ may be used as the lithium salt.

The concentration of the lithium salt may range from about 0.1 M to about 3.0 M. For example, the concentration of the lithium salt may be about 0.5 M or more, and may be about 1.0 M or more. The concentration of the lithium salt may be about 3.0 M or less, about 2.5 M or less, or about 2.0 M or less. In the present disclosure, when the concentration of the lithium salt ranges from about 0.1 M to about 2.0 M, the conductivity of the electrolyte and the viscosity of the electrolyte can be maintained as desired.

### Additive

An additive according to one example embodiment of the present disclosure includes the compound of Chemical Formula 1.

The compound of Chemical Formula 1 may be contained in an electrolyte for a rechargeable lithium battery to provide gas generation reduction and resistance reduction effects in the battery under high voltage and high temperature conditions. In particular, the additive can significantly improve the above gas generation reduction and resistance reduction in a battery including a high nickel-based positive electrode active material having a significantly high nickel content, thereby increasing the lifetime and stability of the battery.

### Compound of Chemical Formula 1

The compound of Chemical Formula 1 is represented by the following Chemical Formula 1. The electrolyte may include one or more compounds of the following Chemical Formula 1.

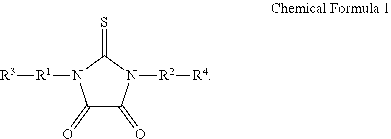

In Chemical Formula 1,
R¹, R², R³, and R⁴ are each as in the above description.

The detailed description of Chemical Formula 1 is omitted because Chemical Formula 1 has been described above.

The compound of Chemical Formula 1 may be included in an amount in a range of about 0.05 wt % to about 5 wt % based on the total amount of the electrolyte. In the above range, the effect of the above mixture can be implemented. For example, the compound of Chemical Formula 1 may be included in an amount in a range of about 0.1 wt % to about 5 wt %, about 0.05 wt % to about 3 wt %, about 0.5 wt % to about 5 wt %, about 2 wt % to about 5 wt %, or about 0.1 wt % to 2 about wt % based on the total amount of the electrolyte. When the content of the additive is within the above range, the effect of the above mixture can be significantly increased, and there can be an additional effect of not increasing the resistance of the battery.

In an example, the compound of Chemical Formula 1 may be included in an amount of about 95 wt % or more, for example, about 95 wt % to about 100 wt %, 99 wt % to 100 wt %, or 100 wt % of the total additives in the electrolyte. In the above range, even without the above additional additive, the battery effect is implemented, and thus the fairness of the battery can be improved.

Accordingly, the electrolyte according to the present disclosure can implement the rechargeable lithium battery in which, by including the additive in the combination of the non-aqueous organic solvent and the lithium salt, it is possible to simultaneously or contemporaneously exhibit the effects of decreasing resistance and reducing gas generation during high-temperature storage in the rechargeable lithium battery including the positive electrode active material, particularly, the positive electrode active material having a high nickel content, thereby improving lifetime characteristics and stability.

Another example embodiment of the present disclosure may include a rechargeable lithium battery including a positive electrode containing a positive electrode active material, a negative electrode containing a negative electrode active material, and an electrolyte, in which the electrolyte contains a non-aqueous organic solvent, a lithium salt, and an additive, and the additive includes the compound of Chemical Formula 1.

The rechargeable lithium battery may be applicable to, e.g., vehicles, mobile phones, and/or various types of electrical devices, and the present disclosure is not limited thereto.

The positive electrode active material may be or include a lithium transition metal composite oxide, and examples thereof include at least one of a lithium nickel-based oxide, a lithium cobalt-based oxide, a lithium manganese-based oxide, a lithium iron phosphate-based compound, a cobalt-free nickel-manganese-based oxide, or a combination thereof.

As an example, a compound represented by any one of the following formulas may be used: LiₐA_{1-b}X_{b}O_{2-c}D_{c} (0.90≤a≤1.8, 0≤b≤0.5, 0≤c≤0.05); LiₐMn_{2-b}X_{b}O_{4-c}D_{c} (0.90≤a≤1.8, 0≤b≤0.5, 0≤c≤0.05); LiₐNi_{1-b-c}Co_{b}X_{c}O_{2-α}D_{α} (0.90≤a≤1.8, 0≤b≤0.5, 0≤c≤0.5, 0<α<2); LiₐNi_{1-b-c}Mn_{b}X_{c}O_{2-α}D_{α} (0.90≤a≤1.8, 0≤b≤0.5, 0≤c≤0.5, 0<α<2); LiₐNi_{b}Co_{c}L¹_{d}GₑO₂ (0.90≤a≤1.8, 0≤b≤0.9, 0≤c≤0.5, 0≤d≤0.5, 0≤e≤0.1); LiₐNiG_{b}O₂(0.90≤a≤1.8, 0.001≤b≤0.1); LiₐCoG_{b}O₂ (0.90≤a≤1.8, 0.001≤b≤0.1); LiₐMn_{1-b}GbO₂ (0.90≤a≤1.8, 0.001≤b≤0.1); LiₐMn₂G_{b}O₄ (0.90≤a≤1.8, 0.001≤b≤0.1); LiₐMn_{1-g}G_{g}PO₄ (0.90≤a≤1.8, 0≤g≤0.5); Li_{(3-f)}Fe₂(PO₄)₃(0≤f≤2); LiₐFePO₄ (0.90≤a≤1.8).

In the above Chemical Formula, A is or includes at least one of Ni, Co, Mn, or a combination thereof, X is or includes at least one of Al, Ni, Co, Mn, Cr, Fe, Mg, Sr, V, a rare earth element, or a combination thereof, D is or includes at least one of O, F, S, P, or a combination thereof, G is or includes at least one of Al, Cr, Mn, Fe, Mg, La, Ce, Sr, V, or a combination thereof, and L¹ is or includes at least one of Mn, Al, or a combination thereof.

The positive electrode active material may include, for example, at least one of a lithium nickel-based oxide represented by the following Chemical Formula 3, a lithium cobalt-based oxide represented by the following Chemical Formula 4, a lithium iron phosphate-based compound represented by the following Chemical Formula 5, a cobalt-free lithium nickel-manganese-based oxide represented by the following Chemical Formula 6, or a combination thereof.

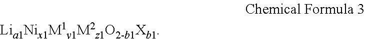

In Chemical Formula 3, 0.9≤a1≤1.8, 0.3≤x1≤1, 0≤y1≤0.7, 0≤z1≤0.7, 0.9≤x1+y1+z1≤1.1, and 0≤b1≤0.1, M¹ and M² each independently is or includes one or more of Al, B, Ba, Ca, Ce, Co, Cr, Cu, Fe, Mg, Mn, Mo, Nb, Si, Sn, Sr, Ti, V, W, and Zr; and X is or includes one or more of F, P, and S.

In Chemical Formula 3, 0.6≤x1≤1, 0≤y1≤0.4, and 0≤z1≤0.4, or 0.8≤x1≤1, 0≤y1≤0.2, and 0≤z1≤0.2.

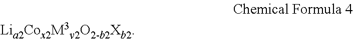

In Chemical Formula 4, 0.9≤a2≤1.8, 0.7≤x2≤1, 0≤y2≤0.3, 0.9≤x2+y2≤1.1, and 0≤b2≤0.1, M³ is or includes one or more of Al, B, Ba, Ca, Ce, Cr, Cu, Fe, Mg, Mn, Mo, Ni, Se, Si, Sn, Sr, Ti, V, W, Y, Zn, and Zr; and X is or includes one or more of F, P, and S.

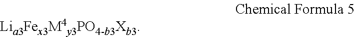

In Chemical Formula 5, 0.9≤a3≤1.8, 0.6≤x3≤1, 0≤y3≤0.4, and 0≤b3≤0.1, M⁴ is or includes one or more of Al, B, Ba, Ca, Ce, Co, Cr, Cu, Mg, Mn, Mo, Ni, Se, Si, Sn, Sr, Ti, V, W, Y, Zn, and Zr; and X is or includes one or more of F, P, and S.

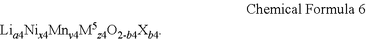

In Chemical Formula 6, 0.9≤a4≤1.8, 0.8≤x4≤1, 0≤y4≤0.2, 0≤z4≤0.2, 0.9≤x4+y4+z4≤1.1, and 0≤b4≤0.1, M⁵ is or includes one or more of Al, B, Ba, Ca, Ce, Cr, Fe, Mg, Mo, Nb, Si, Sn, Sr, Ti, V, W, and Zr; and X is or includes one or more of F, P, and S.

For example, the positive electrode active material may be or include a high nickel-based positive electrode active material having a nickel content of about 80 mol % or more, about 85 mol % or more, about 90 mol % or more, about 91 mol % or more, or about 94 mol % or more and about 99 mol % or less based on 100 mol % of metals excluding lithium in a lithium transition metal composite oxide. The high nickel-based positive electrode active material may implement high capacity and thus may be applied to high-capacity, high-density rechargeable lithium batteries.

In an example embodiment, the negative electrode active material may contain at least one of graphite and a Si composite.

When the negative electrode active material contains a Si composite and graphite together, the Si composite and the graphite may be contained in the form of a mixture, and in this case, the Si composite and the graphite may be contained in a weight ratio in a range of about 1:99 to about 50:50 based on a total of 100 parts by weight. For example, the Si composite and the graphite may be contained in a weight ratio in a range of about 3:97 to about 20:80, about 4:96 to about 20:80, or about 5:95 to about 20:80.

The Si composite includes a core including Si-based particles and an amorphous carbon coating layer, and for example, the Si-based particles may include one or more of a Si—C composite, SiOₓ (0<x≤2), and a Si alloy. For example, the Si—C composite may include a core including Si particles and crystalline carbon, and an amorphous carbon coating layer located on a surface of the core. The crystalline carbon may include, for example, graphite, and for example, may include natural graphite, artificial graphite, or a mixture thereof.

When the positive electrode contains a high nickel-based positive electrode active material having a high nickel content and the negative electrode contains graphite, the effect of improving the high-temperature stability of the rechargeable lithium battery can be improved or maximized.

A driving voltage of the rechargeable lithium battery may be about 4.2 V or higher.

Hereinafter, examples and comparative examples of the present disclosure are described. However, the following examples are merely one example embodiment of the present disclosure, and the present disclosure is not limited to the following examples.

### Synthesis Example 1

10.0 g of 1,3-dicyclohexylthiourea and 50 g of tetrahydrofuran are added to a rounded bottom flask, and stirred at room temperature (25° C.), a solution of 6.34 g of oxalyl chloride dissolved in 20 g of tetrahydrofuran is added dropwise, the temperature is then increased to 60° C., and the mixture is stirred for 12 hours. When the reaction is completed, the temperature is decreased to room temperature, and then 100 g of distilled water is added, and the mixture is stirred for 30 minutes. During the stirring process, a solid is generated and filtered through a filter, washed with distilled water, and then dried in a vacuum oven to obtain a compound represented by the following Chemical Formula 1-1 ((400 MHz, CDCl3):δ 4.63-4.55 (m, 2H), 2.21-2.11 (m, 4H), 1.88-1.69 (m, 10H), 1.38-1.18 (m, 6H)).

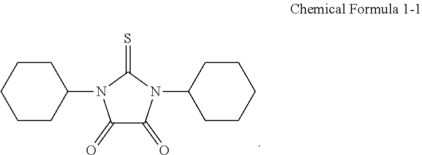 [SMILES: O=C1C(=O)N(C2CCCCC2)C(=S)N1C1CCCCC1]

### Synthesis Example 2

A compound of the following Chemical Formula 1-2 is obtained by the same method as in Synthesis Example 1, except that 1,3-diphenylthiourea is used instead of 1,3-dicyclohexylthiourea ((400 MHz, CDCl3):δ 7.59-7.50 (m, 6H), 7.42-7.37 (m, 4H)).

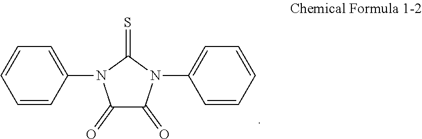 [SMILES: O=C1C(=O)N(c2ccccc2)C(=S)N1c1ccccc1]

### Synthesis Example 3

A compound of the following Chemical Formula 1-3 is obtained by the same method as in Synthesis Example 1, except that 1,3-divinylthiourea is used instead of 1,3-dicyclohexylthiourea ((400 MHz, CDCl3):δ 7.23-7.19 (m, 2H), 5.92 (d, 2H, J=17.1 Hz), 5.21 (d, 2H, J=10.2 Hz)).

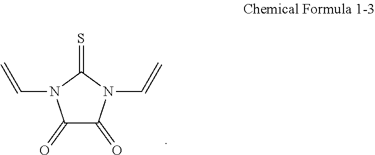

### Synthesis Example 4

A compound of the following Chemical Formula 2 is obtained by the same method as in Synthesis Example 1, except that 1,3-diethylthiourea is used instead of 1,3-dicyclohexylthiourea ((400 MHz, CDCl3):δ 3.68 (q, 4H, J=7.1 Hz), 138 (t, 6H, J=7.1 Hz)).

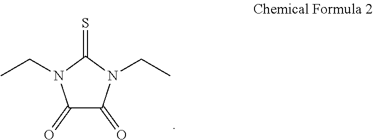

### Examples and Comparative Examples

### Example 1

### (1) Preparation of Electrolyte

An electrolyte was prepared by dissolving 1.0 M LiPF₆ in a carbonate-based solvent containing ethylene carbonate:ethyl methyl carbonate:dimethyl carbonate mixed in a ratio of 20:20:60 (volume ratio) based on a total volume of 100 volumes, adding 0.1 wt % of the compound of Chemical Formula 1-1, and mixing the mixture.

### (2) Manufacturing of Rechargeable Lithium Battery

A positive electrode active material slurry was prepared by mixing 97 wt % of LiNi_{0.91}Co_{0.08}Al_{0.01}O₂ as a positive electrode active material and 0.5 wt % of artificial graphite powder, 1.0 wt % of carbon black (Ketjenblack), and 1.5 wt % of polyvinylidene fluoride (PVdF) as a conductive material, adding the mixture to N-methyl-2-pyrrolidone (NMP), and stirring the mixture for 30 minutes using a mechanical stirrer A positive electrode was manufactured by coating an aluminum current collector having a thickness of 20 μm with the slurry to a thickness of 60 μm using a doctor blade, drying the aluminum current collector in a hot air dryer at 100° C. for 0.5 hours, re-drying the aluminum current collector under vacuum at 120° C. for 4 hours, and roll-pressing the aluminum current collector.

A negative electrode active material slurry was prepared by mixing 98 wt % of a negative active material containing graphite and a Si composite mixed in a weight ratio of 95.8:4.2, 1 wt % of styrene-butadiene rubber (SBR), and 1 wt % of carboxymethyl cellulose (CMC), adding the mixture to distilled water, and stirring the mixture for 60 minutes using a mechanical stirrer. A negative electrode was manufactured by coating a copper current collector having a thickness of 10 μm with the slurry to a thickness of 60 μm using a doctor blade, drying the copper current collector in a hot air dryer at 100° C. for 0.5 hours, re-drying the copper current collector under vacuum at 120° C. for 4 hours, and roll-pressing the copper current collector.

A cylindrical rechargeable lithium battery was manufactured by assembling the positive electrode, the negative electrode, and a separator formed of polyethylene having a thickness of 16 μm to manufacture an electrode assembly and injecting an electrolyte into the electrode assembly.

### Examples 2 to 4

Electrolytes and batteries were manufactured by the same method as in Example 1, except that the content of the compound of Chemical Formula 1-1 was changed as in Table 1 below.

### Example 5

An electrolyte and a battery were manufactured by the same method as in Example 2, except that the compound of Chemical Formula 1-2 prepared from Synthesis Example 2 was used instead of the compound of Chemical Formula 1-1.

### Example 6

An electrolyte and a battery were manufactured by the same method as in Example 2, except that the compound of Chemical Formula 1-3 prepared from Synthesis Example 3 was used instead of the compound of Chemical Formula 1-1.

### Comparative Example 1

An electrolyte and a battery were manufactured by the same method as in Example 1, except that the compound of Chemical Formula 1-1 was not contained.

### Comparative Example 2

An electrolyte and a battery were manufactured by the same method as in Example 1, except that the compound of Chemical Formula 2 prepared from Synthesis Example 4 was used instead of the compound of Chemical Formula 1-1.

### Evaluation Examples

A rechargeable lithium battery was evaluated by the following method.

### Evaluation Example 1: High-Temperature Storage Characteristic Evaluation (DCIR Increase Rate)

For rechargeable lithium batteries according to the examples and comparative examples, initial direct current resistance (DCIR) was measured as a ΔV/ΔI (change in voltage/change in current) value, and then a maximum energy state inside the battery was made into a fully charged state (SOC 100%), and in this state, the battery was stored at 60° C. for 30 days, and then DC resistance was measured, and the DCIR increase rate (%) was calculated according to the following equation, and the results are shown in Table 1 below.

$$DCIR increase rate ( \% ) = ( DCIR after 30 days / initial DCIR ) * 100. \qquad Equation$$

### Evaluation Example 2: Capacity Retention Rate after High-Temperature Storage

The rechargeable lithium batteries of the examples and comparative examples were repeatedly subjected to 0.5C CC/CV charging (4.2 V, 0.05C CUT-OFF) and 0.5C CC discharging (2.8 V CUT-OFF) three times at 25° C. to measure the discharge capacity C1 the third time. The charged rechargeable lithium batteries were stored at 60° C. for 30 days and then were left at room temperature for an additional 30 minutes, and 0.5C CC discharging (2.8 V CUT-OFF) was performed to measure the discharge capacity C2. A capacity retention rate was calculated as follows and is shown in Table 1 below.

$$Capacity retention rate ( \% ) = C 2 / C 1 \times 100 ( \% ) . \qquad Equation$$

### Evaluation Example 3: Gas Generation Rate after High-Temperature Storage

For rechargeable lithium batteries according to the examples and comparative examples, high temperature gas generation characteristic evaluation was performed. To this end, a maximum energy state inside the battery was made into a fully charged state (SOC 100%), and in this state, the gas generation amount (unit: mL) was evaluated in an initial state (before storage at 60° C.) and after 30 days of storage at 60° C. The gas generation rate was measured by measuring a change in a volume before and after high-temperature storage, and converted into a change in a mass using Archimedes' law. The gas increase rate (%) was calculated according to the following Equation, and is shown in Table 1 below.

$$Gas increase rate ( \% ) = ( amount of gas generation after high - temperature storage / amount of gas generation before high - temperature storage ) \times 100 \qquad Equation$$

TABLE 1

| | | | Initial | DCIR | Capacity | Gas |
|---|---|---|---|---|---|---|
| | Compound | | resistance | increase | retention | generation |
| | Type | Content | (mΩ) | rate (%) | rate (%) | rate (%) |
| Example 1 | Chemical | 0.1 | 33 | 132 | 84 | 54 |
| | Formula | | | | | |
| | 1-1 | | | | | |
| Example 2 | Chemical | 0.5 | 35 | 126 | 88 | 48 |
| | Formula | | | | | |
| | 1-1 | | | | | |
| Example 3 | Chemical | 2 | 38 | 118 | 91 | 39 |
| | Formula | | | | | |
| | 1-1 | | | | | |
| Example 4 | Chemical | 5 | 43 | 109 | 92 | 29 |
| | Formula | | | | | |
| | 1-1 | | | | | |
| Example 5 | Chemical | 0.5 | 38 | 130 | 89 | 50 |
| | Formula | | | | | |
| | 1-2 | | | | | |
| Example 6 | Chemical | 0.5 | 37 | 125 | 90 | 45 |
| | Formula | | | | | |
| | 1-3 | | | | | |
| Comparative | — | — | 31 | 150 | 80 | 64 |
| Example 1 | | | | | | |
| Comparative | Chemical | 0.5 | 33 | 149 | 81 | 59 |
| Example 2 | Formula 2 | | | | | |

### Evaluation Example 4: High Temperature (45° C.) Charging/Discharging Cycle Characteristic Evaluation

For rechargeable lithium batteries according to the examples and comparative examples, high temperature charging/discharging characteristic evaluation was performed. To this end, 300 charging/discharging cycles of lithium secondary batteries were performed under the conditions of 45° C., 0.33 C charging (CC/CV, 4.2 V, 0.025 C Cut-off)/1.0 C discharging (CC, 2.8 V Cut-off). The capacity retention rate was calculated according to the following equation, and the results are shown in Table 2 below.

$$Capacity retention rate ( \% ) = ( discharging capacity after 300 cycles / discharging capacity after 1 cycle ) * 100$$

### Evaluation Example 5: DCIR Increase Rate after High Temperature (45° C.) Charging/Discharging Cycle

For rechargeable lithium batteries according to the examples and comparative examples, high temperature charging/discharging characteristic evaluation was performed. After the DCIR was measured as the ΔV/ΔI (change in voltage/change a current) value, the maximum energy state inside the battery was made into a fully charged state (SOC 1000%).

300 charging/discharging cycles of lithium secondary batteries were performed under the conditions of 45° C., 0.33 C charging (CC/CM, 4.2 V, 0.025 C Cut-off)/1.0 C discharging (CC, 2.8 V Cut-off). The DCIR increase rate (0%) was calculated according to the following equation, and the results are shown in Table 2 below.

$$DCIR increase rate ( \% ) = ( DCIR after 300 cycles / initial DCIR ) * 100.$$

TABLE 2

| | | | Initial | DCIR | Capacity |
|---|---|---|---|---|---|
| | Compound | | resistance | increase | retention |
| | Type | Content | (mΩ) | rate (%) | rate (%) |
| Example 1 | Chemical | 0.1 | 33 | 136 | 79 |
| | Formula 1-1 | | | | |
| Example 2 | Chemical | 0.5 | 35 | 129 | 81 |
| | Formula 1-1 | | | | |
| Example 3 | Chemical | 2 | 38 | 122 | 83 |
| | Formula 1-1 | | | | |
| Example 4 | Chemical | 5 | 43 | 117 | 84 |
| | Formula 1-1 | | | | |
| Example 5 | Chemical | 0.5 | 38 | 135 | 81 |
| | Formula 1-2 | | | | |
| Example 6 | Chemical | 0.5 | 37 | 130 | 82 |
| | Formula 1-3 | | | | |
| Comparative | — | — | 31 | 143 | 77 |
| Example 1 | | | | | |
| Comparative | Chemical | 0.5 | 33 | 140 | 78 |
| Example 2 | Formula 2 | | | | |

### SUMMARY

Referring to Tables 1 and 2, it can be determined that the electrolytes of the examples can improve the high-voltage lifetime and high-temperature performance in a rechargeable lithium battery including a high-nickel-based positive electrode active material based on the results of Evaluation Examples 1 to 5.

However, referring to Tables 1 and 2, Comparative Example 1 not including the compound of Chemical Formula 1 of the present disclosure and Comparative Example 2 including the compound of Chemical Formula 2 including compounds other than the compound of Chemical Formula 1 of the present disclosure have relatively high gas generation rates based on the results of Evaluation Examples 1 to 5. Accordingly, compared to the examples, in the rechargeable lithium battery containing the high-nickel-based positive electrode active material, the DCIR increase rate and the gas generation rate are high, and the capacity retention rate is low, and thus it can be determined that the improvement in lifetime under high-voltage and high-temperature performance is significantly insufficient.

The compound and electrolyte according to one example embodiment can exhibit an effect of improving lifetime characteristics and stability under high voltage and high temperature conditions when a rechargeable lithium battery is activated.

Although example embodiments of the present disclosure have been described above, the present disclosure is not limited thereto and may be modified in any form within the scope of the claims, the detailed description of the present disclosure, and the accompanying drawings, and the modifications also fall within the scope of the present disclosure.

What is claimed is:

1. A compound of Chemical Formula 1: 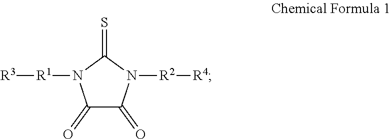
in Chemical Formula 1,
R¹ and R² each independently comprises a single bond, or a substituted or unsubstituted C1 to C5 alkylene group, and
R³ and R⁴ each independently comprises a substituted or unsubstituted C5 to C20 cycloalkyl group, a substituted or unsubstituted C6 to C20 aryl group, a substituted or unsubstituted C7 to C20 arylalkyl group, a substituted or unsubstituted C2 to C20 alkenyl group, or a substituted or unsubstituted C2 to C20 alkynyl group.

2. The compound of claim 1, wherein R¹ and R² each independently comprises a single bond or a methylene group.

3. The compound of claim 1, wherein R³ and R⁴ each independently comprises a substituted or unsubstituted C5 to C10 cycloalkyl group, a substituted or unsubstituted C6 to C10 aryl group, a substituted or unsubstituted C7 to C10 arylalkyl group, a substituted or unsubstituted C2 to C10 alkenyl group, or a substituted or unsubstituted C2 to C10 alkynyl group.

4. The compound of claim 1, wherein the compound of Chemical Formula 1 comprises one or more of the following Chemical Formulas 1-1 to 1-7:

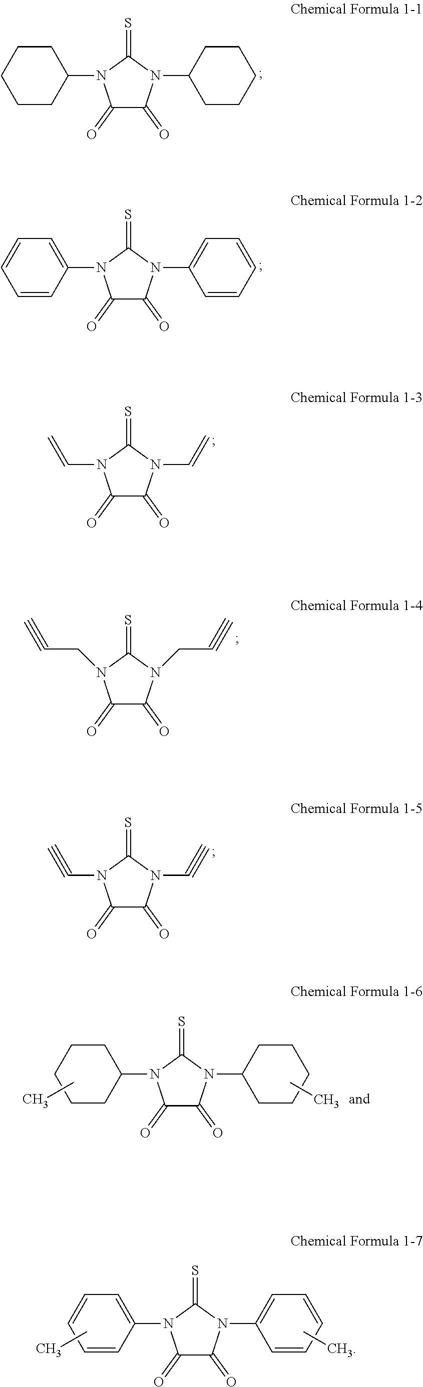

5. An electrolyte for a rechargeable lithium battery, the electrolyte comprising:
a non-aqueous organic solvent;
a lithium salt; and
an additive,
wherein the additive comprises the compound of claim 1.

6. The electrolyte of claim 5, wherein the compound is included in an amount in a range of about 0.05 wt % to about 5 wt % based on a total amount of the electrolyte.

7. The electrolyte of claim 5, wherein the compound is included in an amount of about 95 wt % or more of the additive of the electrolyte.

8. The electrolyte of claim 5, wherein the non-aqueous organic solvent is a mixture including ethylene carbonate (EC):ethyl methyl carbonate (EMC):dimethyl carbonate (DMC) mixed in a volume ratio of about 10 to 30:10 to 30:40 to 80.

9. The electrolyte of claim 5, wherein the lithium salt comprises one or more of LiPF₆, LiClO₄, LiBF₄, lithium bis(fluorosulfonyl)imide (LiFSI), lithium bis(trifluoromethanesulfonyl)imide (LiTFSI), LiSO₃CF₃, LiBOB, LiDFOB, LiDFBOP, LiTFOP, LiPO₂F₂, LiSbF₆, LiAsF₆, LiAlO₂, LiAlCl₄, LiCl, LiI, LiN(SO₃C₂F₅)₂, Li(FSO₂)₂N, and LiC₄F₉SO₃.

10. The electrolyte of claim 5, wherein a concentration of the lithium salt ranges from about 0.1 M to about 2.0 M.

11. A rechargeable lithium battery comprising:
a positive electrode including a positive electrode active material;
a negative electrode including a negative electrode active material; and
the electrolyte of claim 5.

12. The rechargeable lithium battery of claim 11, wherein the positive electrode active material comprises a lithium composite oxide represented by Chemical Formula 3: 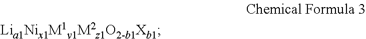
in Chemical Formula 3,
0.9≤a1≤1.8, 0.3≤x1≤1, 0≤y1≤10.7, 0≤z1≤0.7, 0.9≤x1+y1+z1≤1.1, and 0≤b1≤0.1, M¹ and M² each independently comprises one or more of Al, B, Ba, Ca, Ce, Co, Cr, Cu, Fe, Mg, Mn, Mo, Nb, Si, Sn, Sr, Ti, V, W, and Zr, and X comprises one or more of F, P, and S.

13. The rechargeable lithium battery of claim 12, wherein, in Chemical Formula 3, 0.8≤x1≤1, 0≤y1≤0.2, and 0≤z1≤0.2.

14. The rechargeable lithium battery of claim 11, wherein the negative electrode active material comprises at least one of graphite and a Si composite.

15. The rechargeable lithium battery of claim 11, wherein a driving voltage of the rechargeable lithium battery is about 4.2 V or higher.

16. The rechargeable lithium battery of claim 11, wherein the rechargeable lithium battery is one of a cylindrical, prismatic, pouch-shaped, and coin-shaped battery.
